(19) United States
(12) Patent Application Publication
(10) Pub. No.: US 2005/0281766 A1
(43) Pub. Date: Dec. 22, 2005

(54) Method of improving the aesthetic appearance of epithelia

(75) Inventors: Dennis M. Martin, Cornwall, NY (US); Michael Traudt, Brookfield, CT (US); Paul S. Attar, Hawthorne, NJ (US); Isabella L. Morelli-Abrams, Stony Point, NY (US)

Correspondence Address: Charles N. J. Ruggiero, ESQ;OHLANDT, GREELEY, RUGGIERO & PERLE, L.L.P., 10th FLOOR, ONE LANDMARK SQUARE, STAMFORD, CT, 06901-2682, US

(73) Assignee: AVON PRODUCTS, INC.

(21) Appl. No.: 11/175,418

(22) Filed: Jul. 6, 2005

Related U.S. Application Data
(63) Continuation of application No. 10/018,128, filed on Mar. 11, 2002.

(51) Int. Cl. A61K 7/025; A61K 31/203; A61K 31/015

(52) U.S. Cl. 424/64; 514/559; 514/763

(57) ABSTRACT

An effective treatment method for improving the appearance of epithelia, such as lip epithelia and vaginal epithelia is provided. According to the present method, an effective amount of a composition containing retinoid, preferably in a cosmetically acceptable carrier, is topically applied to the vaginal or lip epithelia. The present invention also includes compositions for practicing the method.

This application is a continuation of U.S. Ser. No.10/018,128 filed Mar. 11, 2002, which claims priority in U.S. patent application Ser. No. 09/301,570, filed Apr. 29,1999 and PCT Patent Application Serial No. PCT/US00/11529, filed Apr. 28, 2000.

### BACKGROUND OF THE INVENTION

1. Field Of The Invention

The present invention relates to a method of improving or re-moisturizing the epithelia. More particularly, the present invention relates to a method of improving lip color and reducing the number and depth of lip lines on the surface of the lips, as well as re-moisturizing the vagina and lips. This is achieved by using a composition comprising a retinoid, such as retinol, and a penetration enhancing agent in a cosmetically acceptable carrier. The present invention also relates to a composition, which comprises a retinoid, and to a process for preparing the composition.

2. Description Of The Prior Art

Retinol is known for its beneficial effects in the treatment of acne. In the field of repair of damage caused either by age or overexposure to the sun, retinol has been proven beneficial. Repeated application of cosmetic compositions containing retinol has been used to smooth the skin surface, repair small cracks in the epidermis and to remove wrinkles or minimize the formation thereof.

Because the anatomy and physiology of vagina and the lips differ greatly from the anatomy and physiology of skin proper, the use of retinol for re-moisturizing, treating a color deficiency and/or treating vertical lines, does not have an obvious correlation to skin aging pathologies. As known in the art, vertical lip lines are visually distinguishable from general wrinkling of the lips. As the term “vertical” implies, vertical lip lines appear as substantially vertical creases, whereas “wrinkling” has no such discernible form.

However, it is well known that orally administered retinoids, such as, for example, retinol and retinoic acid, dry out the epithelia and cause cornification of mucosal epithelia.

Research on beneficial effects of retinol has been directed to cosmetic “skin” to produce a reduction in wrinkles and other skin effects related to or resulting from aging. However, there are no reports relating to a method of re-moisturizing the epithelia, improving lip color or reducing the number and depth of lip lines, particularly vertical lip lines, using retinol. Moreover, there are no reports of reversing age-associated cornification of the vaginal or lip epithelia.

Surprisingly, it has been discovered that topically applying a composition including retinoid, such as retinol, and a penetration enhancing agent to mucosal or semi-mucosal epithelium that is dried out and/or cornified, re-moisturizes the epithelium. Furthermore, with repeated application, the cornified epithelia returns to its original mucosal state.

Related U.S. Pat. Nos. 5,656,672 and 5,800,596 provide a water-in-oil emulsion cream, containing retinol, for use as a nourishing and repairing care product for damaged and wrinkled lips.

U.S. Pat. No. 4,826,828 is directed to a water-in-oil emulsion containing retinol, a volatile silicone and a solvent for both the retinol and the volatile silicones. This patent also provides preparation of a retinol emulsion by adding a solution containing retinol to a water-in-oil emulsion. However, to avoid degradation, the retinol is added to the emulsion just prior to or at the time of use. It is apparent that the stability of retinol in a composition of this type is insufficient for prolonged storage prior to use.

U.S. Pat. No. 5,124,313 provides topical compositions having a retinyl ester-polypeptide complex, specifically a retinyl palmitate-polypeptide complex.

WO 93/00085 provides stabilization of retinol in cosmetic compositions by addition to the latter a stabilizing complex comprising, in combination, an antioxidant and a chelating agent for chelating metal ions. The stability of the retinol appears enhanced due to considerable amounts of stabilizing antioxidants and chelating agents in the composition.

WO 97/02814 provides the preparation of an antibacterial medicament containing a retinoid for rapid bactericidal action, particularly on Gram positive bacteria, which accelerates the repair of small lesions.

WO 97/02030 provides cosmetic, antimycotic compositions containing a glycol or glyceryl ester of retinoic acid. These compositions are used to produce visible reduction in wrinkles and visible improvement in tone, firmness and luminosity of skin.

Thus, there as been a need for a topical retinol composition that provides a method for restoring the aged, cornified vaginal epithelia and cornified human lips to their original mucosal state.

### SUMMARY OF THE INVENTION

It is an object of the present invention to provide a method of re-moisturizing epithelia, comprising topically applying to vaginal or lip epithelia an effective amount of a composition comprising a retinoid. The composition can further comprise a cosmetically acceptable carrier.

It is another object of the present invention to provide such a method of re-moisturizing vaginal and lip epithelia in older women, improving lip color, lip clarity and lip dryness, and reducing the number and depth of lip lines.

It is yet another object of the present invention to provide a composition that can be used to re-moisturize vaginal and lip epithelia in older women and improve lip color, lip clarity and lip dryness, and reducing the number and depth lip lines.

These and other objects of the present invention will become apparent in the course of the following description of the preferred embodiments.

### BRIEF DESCRIPTION OF THE DRAWINGS

FIG. 1 is a plot of the average scores for dryness of lips;

FIG. 2 is a plot of the average scores for dry appearance of lips;

FIG. 3 is a plot of the average scores for clarity of lips;

FIG. 4 is a plot of the average scores for color of lips;

FIG. 5 is a plot of the average scores for line quantity of lips; and

FIG. 6 is a plot of the average scores for line depth of lips.

### DETAILED DESCRIPTION OF THE INVENTION

The anatomy and physiology of vagina and the lips of one's mouth differ in many ways from “skin” proper. Because of such differences, the use of a composition having a retinoid, such as retinol, for treating vaginal and lip dryness or treating color deficiency and/or lines of the lip is not an obvious correlation to the treatment of aging pathologies associated with other types of skin. Thus, the present invention is the first to demonstrate a clinical benefit when applied topically to a vaginal epithelium and to a semi-mucosal epithelium, which is within and up to the vermilion border of the lips. In addition, the present invention is the first to demonstrate that the aesthetic appearance of epithelia is improved by applying a topical composition having both a retinoid, particularly retinol, in an amount effective to improve the aesthetic appearance of the epithelial and a penetration enhancing agent in an amount effective to enhance penetration of the retinoid into the epithelia. The applicants have unexpectedly discovered an effective method for improving human lip color (i.e., increasing redness) and reducing the number and depth of lip lines (i.e., line quantity), as well as re-moisturizing vaginal and lip epithelia in older women. As used in the context of the present invention, the term “older” refers to post-menopausal women and/or women who suffer from age-related vaginal and lip dryness or cornification.

When used as a vaginal treatment for re-moisturizing vaginal epithelia, the method of the present invention must be applied almost exclusively to older women who are experiencing routine vaginal dryness. The composition is preferably applied daily to inner surfaces of the vagina, before bedtime.

Without being bound by any theory, it is believed that, as they age, epithelial cells may lose responsiveness to circulating retinoids, such as retinol, causing a fundamental change in the structure of the cells, i.e., squamous metaplasia. With a supply of excess retinoid available to the cells, the cells respond and return to their mucosal state. The penetration enhancing agent provides improved delivery of the retinoid to the “active” site located at the epithelial cell.

A beneficial re-moisturizing effect is obtained on repeated application of the composition to the vaginal epithelia. To produce the beneficial re-moisturizing effect of the method of the present invention, the composition is preferably applied to the vaginal epithelia in the form of a cream.

On repeated application of the composition to the lips, particularly aging lips, in accordance with the method of the present invention, a visible improvement in lip condition, moisturization, color, clarity and a measurable reduction in the number and depth of lip lines is observed within a short period of time, which can be as little as two (2) weeks, when applied once or twice a day. The improvement in the color of the lips is manifested by an increase in the redness of the lips, whereas the reduction in the number of lines is directly measured and reduction in the dryness and depth of the lines estimated by direct observation as described below.

In the context of the present invention, the term retinoid includes the following classes of compounds: retinol; esters of retinol with carboxylic acids of 1 to 24 carbon atoms, such as retinyl acetate, retinyl propionate, retinyl butyrate, retinyl octanoate, retinyl laurate, retinyl palmitate, retinyl oleate, retinyl linoleate; esters of retinol with alpha-hydroxy carboxylic acids; ether derivatives of retinol, including alkyl ethers, ethers derived from glycolic acid and glycolate esters and amides, such as retinyl glycolyl ether (retinyl glycolic acid ether); retinaldehyde; retinoic acid; esters of retinoic acid with alcohols of 1 to 24 carbon atoms; isotretinoin as well as synthetic retinoid mimics, and derivatives of the foregoing, as well as others that bind to RAR receptors; cis- and trans-isomers thereof; salts thereof; and mixtures thereof. Preferably, the retinoid is retinol. More preferably, the retinoid is the trans-isomer of retinol. Retinol may be prepared by well-known methods such as those described in U.S. Pat. No. 3,060,229, the content of which is incorporated herein by reference.

Retinyl esters, which generally are less potent than other retinoids, are less preferred retinoids for the purposes of the present invention. Specifically, retinyl ester-polypeptide complexes, such as those described in U.S. Pat. No. 5,124,313, are not contemplated as retinoids within the context of the present invention.

The amount of retinoid in the composition of the present invention is preferably in the range from about 0.001 to about 1.5 weight percent (wt %) of the total composition, more preferably from about 0.06 wt % to about 0.3 wt %, and most preferably about 0.1 wt % to about 0.2 wt % of the composition. However, as stated above, the amount of retinoid may be adjusted, based upon the potency of the retinoid, without departing from the present invention.

The penetration enhancing agent is present in an amount effective to either enhance the penetration of the selected retinoid into the epithelia or increase the rate (i.e., speed) of penetration of the selected retinoid into the epithelia. Preferably, the penetration enhancing agent does both. The selection of the penetration enhancing agent will depend on formulation factors, such as the chemical properties of the selected retinoid and the vehicle (e.g., solution, emulsion, stick). Non-limiting examples of such penetration enchancing agents include: organic solvents, such as ethanol, glycols (e.g., propylene, butylene, pentylene), pyrrolidones (e.g., 2-pyrrolidone, 1-methyl-2-pyrrolidone, 5-methyl-2-pyrrolidone, 1,5-dimethyl-2-pyrrolidone, 1-ethyl-2-pyrrolidone, 2-pyrrolidone carboxylic acid), dimethyl sulfoxide, dimethylacetamide, and dimethylformadide); alkyl sulfoxide; phosphine oxide; sugar esters (e.g., sucrose acetate, sucrose octanoate); anionic surfactants; nonionic surfactants; Azone™ (e.g., 1-dodecylazaclo-heptan-2-one); N-substituted di-isopropanolamines; fatty acids (e.g., oleic acid, linoleic acid); and mixtures thereof. Additional resources are available to those in the art to assist with the selection of the penetration enhancing agent. One such resource is available at pages 160 through 172 of Dermatological Formulations, B. W. Barry (Marcel Decker, 1983), which is incorporated herein by reference.

In addition to the retinoid and the penetration enhancing agent, the composition of the invention can contain a cosmetically acceptable carrier. The carrier can be water, a humectant, a thickener, a gelling agent, an oil, an emulsifier, or mixtures thereof. The resulting composition can be in the form of a cream, dispersion, emulsion, foam, gel, solution, stick suspension, spray, patch, powder or in a towelette. The emulsion can be either an oil-in-water emulsion or a water-in-oil emulsion.

Preferably, the retinoid is formulated into an appropriate vehicle consistent with consumer requirements. For example, the composition can preferably be formulated as a stick or cream for treatment of the lips. For the treatment of the vaginal epithelia, the composition would preferably be formulated as a cream.

The oil phase of the emulsion preferably has one or more organic compounds, including emollients. The aqueous phase has humectants, such as propylene glycol and glycerin; other water-dispersible or water-soluble components including thickeners such as veegum or hydroxyalkyl cellulose; gelling agents, such as high MW polyacrylic acid, i.e. carbopol 934; and mixtures thereof. The emulsion has one or more emulsifiers capable of emulsifying the various components present in the composition, including the retinoids. Because of the light, heat and air sensitivity of retinoids, retinoids are generally added last in the preformed emulsion so as to minimize exposure to light, heat and oxygen.

Non-limiting examples of organic compounds suitable for use in the oil phase include emollients, skin conditioning agents, solvents that are capable of dissolving retinol or retinoids without reducing the stability thereof, and mixtures thereof. The compounds suitable for use in the oil phase include one or more of the following: an alcohol including cetyl alcohol; ester including cetyl recinoleate, sterol esters; carboxylic acid; mineral oil; wax; hydrocarbon; paraffin; isoparaffin; petrolatum; low taste petrolatum for application on lips; hydrogenated polydecene; silicone-containing compound such as dimethyl polysiloxane; and mixtures thereof.

The emulsifier can be an emulsifying wax, an emulsifying polyhydric alcohol, a polyether polyol, a polyether, a mono- or di-ester of a polyol, an ethylene glycol mono-stearate, a glycerin mono-stearate, glycerin di-stearate, a silicone-containing emulsifier, a soya sterol, a fatty alcohol such as cetyl alcohol, a fatty acid such as stearic acid, a fatty acid salt, and mixtures thereof. The preferred emulsifiers include soya sterol, cetyl alcohol, stearic acid, emulsifying wax, and mixtures thereof.

The emulsifying waxes that are suitable for use in the present invention are well known to persons skilled in the art. The emulsifying wax includes compositions such as those containing about 80 wt % cetearyl alcohol, about 10 wt % polysorbate 60, about 5 wt % stearate, and about 5 wt % steareth-20.

In the emulsion, the aqueous phase is preferably from about 60 wt % to about 90 wt %, the oil phase is preferably from about 5 wt % to about 30 wt % of the emulsion, and the emulsifier is preferably from about 5% to about 10 wt % of the emulsion.

The emulsion according to the present invention has pH preferably in the range from about 6.5 to about 7.5.

The composition according to the present invention can be prepared by dissolving a retinoid, such as retinol, in a medium comprising an organic solvent, and optionally water, and adding the resulting homogeneous solution to the emulsion. The composition produced thereby preferably has from about 0.001 wt % to about 1.0 wt % retinol, on an active basis, and about 0.5 wt % to about 1.0 wt % organic solvent. More preferably, the composition has from about 0.06 wt % to about 0.3 wt % retinol.

The present invention includes a process for preparing a composition comprising a retinoid, such as retinol, in the form of a cream, dispersion, emulsion, foam, gel, solution, stick or suspension.

The process for preparing a composition for re-moisturizing epithelia, comprises:
preparing a first mixture comprising water, a humectant, a thickener, and a gelling agent;
preparing a second mixture comprising an oil and an emulsifier;
adding the second mixture and the first mixture together, preferably mixing the second mixture in the first mixture, at a temperature and period of time sufficient to produce a stable emulsion;
cooling the stable emulsion; and
adding a retinoid to the stable emulsion to produce the composition.

In addition, the present invention may include a secondary component. The secondary component is preferably selected from one or more of the following thirteen groups.

1. Rexinoids: Rexinoids include compounds, such as all-trans retinoic acid, 9-cis retinoic acid, phytanic acid and others, that bind to RXR receptors.

2. An estrogen synthetase (aromatase) stimulating compound: Examples of such a compound include caffeine and/or derivatives thereof, and any mixture thereof. Caffeine is the more preferred of such compounds.

3. A compound capable of inhibiting 5 alpha-reductase activity: Examples of such a compound include linolenic acid, linoleic acid, finasteride, and mixtures thereof.

4. An exfoliation promoting compound: Suitable examples include alpha hydroxy acids; beta hydroxy acids; oxa acids as disclosed in U.S. Pat. No. 5,847,003 (the disclosure of which is incorporated herein by reference); oxa diacids as disclosed in U.S. Pat. No. 5,834,513 (the disclosure of which is incorporated herein by reference); mechanical exfoliation compounds, such as bamboo exfoliant extract; salicylic acid; benzoyl peroxide; keto acids, such as pyruvic acid, 2-oxopropanoic acid, 2-oxobutanoic acid, and 2-oxopentanoic acid; and mixtures thereof.

The preferred exfoliation promoting compounds are lactic acid, glycolic acid, 3,6,9-trioxaundecanedioic acid, and any mixture thereof. When the present invention includes an exfoliation promoting compound, the composition comprises about 1 wt % to 20 wt %, preferably about 1 wt % to about 15 wt %, more preferably about 4 wt % to about 10 wt % acid, and most preferably about 4 wt %, of the exfoliation promoting compound.

5. An ultraviolet (UV) light protecting/sunscreen agent: Examples include organic and inorganic sunscreens, such as titanium dioxide, zinc oxide, methyl benzylidene camphor and/or its derivatives, octocrylene, anthranilates, benzophenones, butylmethoxydibenzoylmethane (avobenzone), naphtholsulphonates, benzoic acid derivatives, salicylates, cinnamic acid derivatives, terephthalylidene dicamphor sulfonic acids, and mixtures thereof. Of these, butylmethoxydibenzoylmethane, octocrylene, octylsalicylate, octylmethoxycinnamate, oxybenzone, titanium dioxide, and mixtures thereof are preferred. Butylmethoxydibenzoylmethane, oxybenzone, octylmethoxycinnamate, terephthalylidene dicamphor sulfonic acids, and mixtures thereof are most preferred. Salts, esters and other derivatives of the aforementioned sunscreen agents, which are compatible with the composition, are also contemplated in practicing the present invention. A preferred UV absorber includes a hydroxybenzophenone derivative, a benzotriazole derivative, a dibenzoyl methane derivative, an oxanilide derivative, a hydroxy cinnamate derivative, and mixtures thereof.

Co-formulation with an ultraviolet light protecting/sunscreen agent is particularly desirable when the present invention is used for treating lip epithelia, particularly for users who engage in activities, particularly outdoor activities, which expose the user's lip epithelia to UV radiation. Non-limiting examples of such activities include indoor tanning, sunbathing, and skiing. It is preferred that the sunscreen comprises from about 2 wt % to about 20 wt %, more preferably about 2 wt % to about 15 wt %, of the total weight of the composition.

6. Barrier function enhancing agents: Examples include ceramides; essential fatty acids and their esters, especially glycerides, α-hydroxy fatty acids and their esters, ω-hydroxy fatty acids and their esters; phospholipids; cholesterol and its esters, such as cholesteryl hemisuccinate, cholesteryl phosphate; and cholestanol and its derivatives. The barrier function enhancing agent can be added to a topical composition either as singular molecular entities or as a complex mixture of lipids derived from either synthetic, animal or plant sources.

7. Collagen enhancing agents: These agents prevent epithelia “sagging” by promoting a net increase in collagen, either by reducing collagen breakdown or by promoting collagen formation. Examples of such agents include Clara extract (Sophora augustifolia), ascorbyl-phoshoryl-cholesterol, ascorbic acid, ascorbic acid derivatives, and mixtures thereof.

8. Elastase inhibitors: Examples of these inhibitors include fatty acids, such as oleic acid, perinaric acid, and Honeysuckle extract (Lonicera caprifolium). These inhibitors act to prevent sagging of the epithelia.

9. Skin lightening agents: Examples include kojic acid, hydroquinone, licorice derivatives, ascorbic acid/ascorbic acid derivatives (e.g. magnesium ascorbyl phosphate), arbutin, bearberry (Arctostaphylos uva ursi), Glycyrrhiza glabra and its derivatives, Chlorella vulgaris extract, and mixtures thereof.

10. Antioxidants: Examples include compounds having phenolic hydroxy functions, such as ascorbic acid, ascorbic acid derivatives; gallic acid derivatives (e.g. propyl gallate); ferulic acid derivatives (e.g. ethyl ferulate, sodium ferulate); nitrones; N-tertbutyl-nitrone; I-(4-pyridyl-1-oxide)-N-tertbutyl-nitrone; curcumin, tetrahydrocurcumin; 6-hydroxy-2,5,7,tetramethylchroman-2-carboxylic acid; uric acid; reductic acid; tannic acid; rosmarinic acid; tocopherol and its derivatives; catechins; and mixtures thereof. Other suitable antioxidants are those that have one or more thiol functions (—SH), in either reduced or non-reduced form, such as glutathione, lipoic acid, thioglycolic acid, and other sulfhydryl compounds. The antioxidant may be inorganic, such as sulfites, bisulfites, metabisulfite, or other inorganic salts and acids containing sulfur. Preferably, the antioxidant is selected from the group consisting of: a phenolic antioxidant such as butylated hydroxytoluene; butylated hydroxyanisole; an alkyl paraben such as methyl, ethyl or propyl paraben; and any mixtures thereof.

11. Skin warming agents: Examples include vanillyl butylamid, capsaicin, and mixtures thereof.

12. Skin cooling compounds: Examples include menthol, menthyl glycerol, asymmetrical carbonates, thiocarbonates and urethanes, N-substituted carboxamides, ureas or phosphine oxides, menthyl lactate, menthone glycerine acetal, and any mixtures thereof.

13. Anti-pruretic/Anti-itch compounds: Non-limiting examples of such compounds include capsaicin, nonivamde, and corticosteroids. Co-formulation with an anti-pruretic/anti-itch compound may be desirable when the present invention is applied to itchy vaginal epithelia. A non-limiting example of when such co-formulation may be desirable includes when the user has a concurrent condition commonly referred to as a yeast (Candida albicans) infection.

The addition of the secondary component enhances the dermatological benefits achieved and the utilization for compositions of the present invention. The compositions of the present invention may include at least two secondary components, with each secondary component being selected from a different group.

The compositions of the present invention can include other cosmetic and pharmaceutical actives and excipients. Such suitable cosmetic and pharmaceutical agents include, but are not limited to, one or more of erythromycins, tetracyclines, salicylic acids, antifungals, vitamins, anti-inflammatory agents, antimicrobials, analgesics, nitric oxide synthase inhibitors, self-tanning agents, surfactants, moisturizers, stabilizers, preservatives, antiseptics, chelating agents, thickeners, emulsifiers, lubricants, humectants, chelating agents, skin penetration enhancers, skin cooling agents, emollients, fragrances, colorants, flavoring agents, pigments, and mixtures thereof.

Other conventional constituents including cosmetic and pharmaceutical additives may be added to the composition. These additives include: vitamins, such as tocopherol and ascorbic acid; vitamin derivatives such as ascorbyl monopalmitate; thickeners such as hydroxyalkyl cellulose; gelling agents; structuring agents such as bentonite, smectite, magnesium aluminum silicate and lithium magnesium silicate; metal chelating agents such as EDTA; pigments such as zinc oxide and titanium dioxide; colorants; emollients; and humectants.

When the present invention is used to improve the aesthetic appearance of lip epithelia, the compositions of the present invention may be non-pigmented or pigmented. Lip compositions, such as lipsticks, often have pigments incorporated therein. An example of a pigment is iron oxides. However, when the composition is pigmented, it is preferred that the composition includes an ascorbyl-phosphoryl-cholesterol (APC) compound. Examples of suitable APC compounds are disclosed in WO 97/42960, which is commonly assigned and is incorporated herein by reference.

The addition of an APC compound in pigmented compositions of the present invention is particularly desirable when the pigment is an iron oxide. An example of such a pigmented composition may have from about 0.2 wt % to about 20 wt % of iron oxides in addition to the APC compound. One preferred example of such a topical composition has from about 5 wt % to about 7 wt % of iron oxides and about 1 wt % of the APC compound in a suitable vehicle. In the preferred example, the iron oxides are selected from the group consisting of iron oxide red 2259-preserved, iron oxides (yellow), iron oxides (black), and mixtures thereof. Additional advantages of including an APC compound are set forth in PCT WO 00/06091, which is commonly assigned and is incorporated herein by reference.

When the present invention is applied to lip epithelia, particularly in the form of a pigmented composition, it is preferred that the weight percentage of retinoid in the pigmented composition is adjusted to accommodate numerous re-application as may occur with topical lip compositions, such as lipsticks and lip balms.

Compositions of the present invention can be applied to epithelia for a period of time to improve the aesthetic appearance of the epithelia. The improvement in aesthetics can include at least one of the following:
a. reducing intrinsic aging;
b. reducing photoaging;
c. decreasing epithelial fragility;
d. preventing and reversing loss of collagen;
e. preventing epithelial atrophy;
f. promoting/accelerating cell turnover;
g. improving epithelial firmness/plumpness;
h. improving epithelial texture;
i. decreasing fine lines;
j. decreasing wrinkles;
k. improving epithelial tone;
l. enhancing epithelial thickness;
m increasing moisture retention;
n. minimizing epithelial discoloration; and
o. reversing age-associated cornification of epithelia.
   Other improvements in the aesthetic appearance of epithelia are provided by the present invention. The above improvements are only examples of the improvements made possible by the present invention and are set forth for illustration only.

The Examples that follow are intended for illustrating the present invention and not for limiting the scope thereof.

Clinical Study Results

Efficacy of retinol-containing creams in reducing visible signs of aging lips was demonstrated as follows.

Two lip creams, Lip Cream A containing 0.15% active retinol and Lip Cream B containing 0.30% active retinol were prepared as shown below in TABLE 1.

TABLE 1
Preparation Of Lip Cream A And Lip Cream B

| INGREDIENTS (Wt %) | | |
|---|---|---|
| Retinol Cream | LIP CREAM A | LIP CREAM B |
| Retinol | 0.30 | 0.15 |
| Acrylates Copololymer | 1.10 | 0.55 |
| Carbopol/thickeners | 0.90 | 0.90 |
| Disodium EDTA | 0.20 | 0.20 |
| Glycerin | 5.00 | 5.00 |
| Propylene Glycol | 0.56 | 0.56 |
| Emollients | 13.50 | 13.50 |
| Emulsifiers | 8.50 | 8.50 |
| Preservatives | 1.40 | 1.40 |
| Anti-oxidants | 0.10 | 0.10 |
| Triethanolamine | 1.00 | 1.00 |
| Demineralized Water | qs | qs |

The study was a double blind monadic design. A total of 36 female subjects who met the inclusion criteria ranging in age from 33 to 64 years were selected for this study. The subjects were in good general health, with no known allergies or sensitivities to lip products. The subjects had skin types I-III (predominantly I-II), were not pregnant or nursing, had full lips, exhibiting acceptable appearance of aging lips, including paleness, mild dryness and flaking of the surface, and some vertical lines, but not premature aging.

Each subject's lips were visually examined (baseline examination) and signs of dryness, flaking, paleness, fullness and lip lines were recorded.

The subjects were randomly divided into two groups and each group was asked to use one Lip Cream once a day, at night, shortly before bedtime, after cleansing their face prior to using the Lip Cream. They were asked to apply a small amount of product onto a finger and spread the product on both the upper and lower lip, avoiding the outer edges of the lips, with the lips closed, to avoid getting the product into the mouth. The use of usual lip products such as lipstick and/or lip balm was permitted except on examination days.

At baseline the following visual parameters were graded (5-point subjective scoring):
(1) Dryness (flaking): 0=no visible flakes, 5=severe flaking;
(2) Dry appearance (visible tightness): 0=none, 5=severe;
(3) Color: 0=very pale, 5=dark red;
(4) Clarity (transparency): 0=clear, 5=highly opaque;
(5) Number of lines: 0=none, 5=numerous; and
(6) Depth of lines: 0=shallow, 5=deep.

Lip Cream A and Lip Cream B were then dispensed and use instructions administered. Briefly, subjects were instructed to apply the test product to their lips once a day, before bedtime.

Subjects returned for follow-up visual grading after 2, 4 and 8 weeks. In addition, 35 mm frontal lip photos were taken at baseline and after 8 weeks. Finally, a self-perception questionnaire was administered at 8-weeks.

TABLE 2 compares the average visual scores of the two treatment groups.

TABLE 2
Efficacy of Retinol-Containing Creams in
Reducing Visible Signs of Aging Lips
Average Visual Scores

| | | FORMULA A | FORMULA B |
|---|---|---|---|
| | | 0.3% Retinol | 0.15% Retinol |
| Feature Evaluated | Week | Mean (N = 18) | Mean (N = 18) |
| Dryness | 0 | 1.00 | 1.06 |
| | 2 | 1.09 | 1.08 |
| | 4 | 0.44 | 0.56 |
| | 8 | 0.34 | 0.58 |
| Dry Appearance | 0 | 2.41 | 2.58 |
| | 2 | 2.44 | 2.53 |
| | 4 | 1.63 | 1.83 |
| | 8 | 1.09 | 1.36 |
| Clarity | 0 | 3.84 | 3.86 |
| | 2 | 3.53 | 3.58 |
| | 4 | 2.69 | 2.89 |
| | 8 | 1.91 | 2.00 |
| Color | 0 | 1.84 | 1.86 |
| | 2 | 2.06 | 2.06 |
| | 4 | 2.69 | 2.50 |
| | 8 | 3.09 | 2.83 |
| Number of Lines | 0 | 3.22 | 3.31 |
| | 2 | 3.09 | 3.17 |
| | 4 | 2.75 | 2.89 |
| | 8 | 2.31 | 2.47 |
| Line Depth | 0 | 1.84 | 2.03 |
| | 2 | 1.69 | 1.75 |
| | 4 | 1.50 | 1.67 |
| | 8 | 1.28 | 1.50 |

From the results obtained, it can be seen that:
(1) there was no statistical difference between the two treatment groups;
(2) both Lip Creams were clinically well tolerated without any adverse clinical repsonse attributable to the use of Lip Cream A or Lip Cream B;
(3) all parameters improved after 8 weeks;
(4) most parameters improved at 4 weeks, except number of lines, dryness and the 0.15% retinol (Formula B) line depth score; and
(5) the percent improvement, calculated from the average scores within an interval, was always numerically superior for Lip Cream A containing 0.3% retinol than for Lip Cream B containing 0.15% retinol.

Calculation of percent improvement for Lip Cream A containing 0.3% retinol revealed a 68% improvement in lip color and 28% reduction in the number of vertical lip lines and 30% reduction in the depth of vertical lip lines within 8 weeks of treatment.

While the difference in efficacy between Lip Cream A and Lip Cream B was not large, the magnitude of improvements in dryness, dry appearance, color, clarity and lines of the lips obtained from Lip Cream A containing 0.3% retinol was greater for all measured parameters. In addition, both Lip Cream A and Lip Cream B were clinically well tolerated.

FIGS. 1 through 6 chart the average scores for each parameter. These graphs demonstrate that, for all parameters, the most dramatic improvement was observed between 2 and 4 weeks of Lip Cream use.

Features associated with aging lips, including dryness (flaking/taut), dry appearance, color, clarity and number and depth of lines were all significantly improved by the use of the retinol-containing Lip Cream A and Lip Cream B according to the present invention.

Obvious modifications and variations of the present invention are possible in light of the above teachings. It is therefore to be understood that such modifications not specifically described herein are within the scope of the appended claims. Also, singular used in the application can also mean plural of the same ingredient.

1. A method of improving the aesthetic appearance of epithelia comprising:
applying to the epithelia a topical composition comprising:
a retinoid in an amount effective to improve the aesthetic appearance of the epithelia; and
a penetration enhancing agent in an amount effective to enhance penetration of said retinoid into the epithelia,
wherein said topical composition is applied to the epithelia for period of time effective to provide the improvement.

2. The method of claim 1, wherein the epithelia is selected from the group consisting of lip epithelia and vaginal epithelia.

3. The method of claim 2, wherein the epithelia is lip epithelia.

4. The method of claim 1, wherein the improvement in aesthetic appearance is a reduction in the appearance of aging of the lips.

5. The method of claim 4, wherein the aging of the lips is photoaging or intrinsic aging.

6. The method of claim 4, wherein the improvement in aesthetic appearance is selected from the group consisting of:
a. improvement in lip color;
b. improvement in lip dryness;
c. improvement in lip clarity;
d. reduction in the number vertical lip lines;
e. reduction in the depth of vertical lip lines;
f. improvement in lip dryness appearance; and
g. combinations thereof.

7. The method of claim 1, wherein said retinoid is in amount from about 0.001 wt % to about 1.5 wt % of the total weight of the composition.

8. The method of claim 1, wherein said retinoid is retinol.

9. The method of claim 1, wherein the penetration enhancing agent is selected from the group consisting of: an organic solvent, an alkyl sulfoxide, a phoshine oxide, a sugar ester, a anionic surfactant, a non-ionic surfactant; an Azone, a N-substituted di-isopropanolamine, a fatty acid alcohol, and mixtures thereof.

10. The method of claim 1, wherein the penetration enhancing agent is selected from the group consisting of ethanol, propylene glycol, butylene glycol, pentylene glycol, 2-pyrrolidone, 1-methyl-2-pyrrolidone, 5-methyl-2-pyrrolidone, 1,5-dimethyl-2-pyrrolidone, 1-ethyl-2-pyrrolidone, 2-pyrrolidone carboxylic acid, dimethyl sulfoxide, dimethylacetamide, dimethylformadide; alkyl sulfoxide; phosphine oxide; sucrose acetate, sucrose octanoate, 1-dodecylazaclo-heptan-2-one, oleic acid, linoleic acid,and mixtures thereof.

11. The method of claim 1, wherein the composition further comprises a cosmetically acceptable vehicle.

12. The method of claim 11, wherein the vehicle is selected from the group consisting of: an emulsion, a gel, and a stick, a suspension, a foam, a stick, a solution, a spray, a patch, a powder and a towelette.

13. The method of claim 1, wherein the composition has a pH less than about 7.5.

14. The method of claim 11, wherein the vehicle is anhydrous.

15. The method of claim 1, wherein said topical composition further comprises a secondary component selected from the group consisting of:
a. a rexinoid;
b. an estrogen synthetase (aromatase) stimulating compound;
c. a 5 alpha-reductase activity inhibitor;
d. an exfoliation promoting compound;
e. an ultraviolet (UV) light protecting/sunscreen agent;
f. a barrier function enhancing agent;
g. a barrier function enhancing agent;
h. an elastase inhibitor;
i. a skin lightening agent;
j. an antioxidant;
k. a skin warming agent;
l. a skin cooling compound; and
m. an anti-pruretic/anti-itch compound.

16. The method of claim 15, wherein the secondary component is said sunscreen.

17. The method of claim 16, wherein said sunscreen is selected from the group consisting of:
a. avobenzone;
b. octylmethoxycinnamate;
c. oxybenzone;
d. titanium dioxide;
e. octyl salicylate; and
f. mixtures thereof.

18. A composition for improving the aesthetic appearance of epithelia comprising:
a retinoid in an amount effective to improve the aesthetic appearance of the epithelia; and
a penetration enhancing agent in an amount effective to enhance penetration of said retinoid into the epithelia.

19. The composition of claim 18, further comprising a sunscreen agent selected from the group consisting of:
a. avobenzone;
b. octylmethoxycinnamate;
c. titanium dioxide;
d. octyl salicylate; and
e. mixtures thereof.
